(19) United States
(12) Patent Application Publication
(10) Pub. No.: US 2009/0177229 A1
(43) Pub. Date: Jul. 9, 2009

(54) SUTURE ANCHOR WITH DRUG/GROWTH FACTOR DELIVERY RESERVOIR

(75) Inventors: Lawrence V. Gulotta, New York, NY (US); Darrick Lo, Green Brook, NJ (US); Joseph David Lipman, New York, NY (US)

Correspondence Address: DARBY & DARBY P.C., P.O. BOX 770, Church Street Station, New York, NY, 10008-0770, US

(73) Assignee: New York Society of the Ruptured and Crippled Maintaining the Hospital for Special Surgery, New York, NY (US)

(21) Appl. No.: 12/344,749

(22) Filed: Dec. 29, 2008

Related U.S. Application Data
(60) Provisional application No. 61/018,791, filed on Jan. 3, 2008.

(51) Int. Cl. A61B 17/04; A61M 37/00

(52) U.S. Cl. 606/232; 604/93.01; 604/500

(57) ABSTRACT

A surgical device for use in attaching a first member (e.g., soft-tissue) to a second member (e.g., a bone) includes a suture anchor that has a body having a first end; a pointed tip at an opposite second end; and a contoured outer surface. An anchor head that is coupled to the body at the first end has an opening to permit attachment of one or more suture to the body. The suture anchor also includes a reservoir formed in the body and being open at the first end thereof and closed at an opposite end. The reservoir is configured to store and release at least one of a drug, a therapeutic agent, a growth factor, or a combination thereof from a top surface of the anchor body into a site of an interface between the first and second members to promote healing or therebetween or limit post-operative pain.

This application claims priority to U.S. Provisional Application No. 61/018,791, filed Jan. 3, 2008. The contents of this provisional application are hereby incorporated by reference in their entirety.

### TECHNICAL FIELD

The present invention relates generally to a process and device or assembly for use in tissue repair and in particular, relates to a suture anchor that has a reservoir that contains a drug, a growth factor or a therapeutic agent or combination thereof and is configured to optimize healing at the bone/tendon interface and/or prevent post-operative pain.

### BACKGROUND

Soft tissues, such as tendons and ligaments, generally are attached to a bone by small collagenous fibers. These connections are strong but permit the tendons and ligaments to be flexible. When a tissue, or a portion of a tissue, is torn away from the bone and requires repair, a surgeon is often required to repair the detached soft tissue with sutures which are passed through bone tunnels and tied. There are a number of different types of devices that are designed for securing soft tissue, such as ligaments, tendons, muscles, as well as objects, such as prostheses to bone. For example, an object can be attached to a bone using screws, staples, cement, suture anchors, and sutures alone.

A suture anchor assembly utilizes small anchors with suture materials attached thereto. A device, such as a screw, is inserted into the bone mass and anchored in place. After insertion of the screw, the attached suture is passed through the tissue to be repaired. The suture is then tied in a knot to secure the tissue to the bone.

Suture anchors are commonly used in orthopedic surgery to repair soft tissues to bone. One example of their use is in rotator cuff repair surgery. Clinical studies have shown that surgically repaired rotator cuffs fail to heal to the bone anywhere from 40-90% of the time, depending upon the nature of the tear. While many patients remain pain-free even when the cuff fails to heal, recent studies have shown that these patients have inferior functional results when compared to patients who have successfully healed. It has been appreciated that the tendon heals to the bone through a scar tissue interface that represents a “weak link” in the repaired construction. As a result, it is desirable to improve tendon-to-bone healing in an effort to reduce repair failures. One technique is to use bone morphogenetic proteins (BMPs), which are a group of growth factors known for their ability to induce the formation of bone and cartilage. For example, BMP2 acts as a disulfide-linked homodimer and induces bone and cartilage formation and plays a key role in osteoblast differentiation. BMP7 also play a key role in osteoblast differentiation and it also induces the production of SMAD1.

Most commercially available growth factors are supplied on a carrier that consists of either a collagen sponge (BMP-2, Medtronic; BMP7, Stryker); a ceramic carrier (PDGF-BB, BioMimetics); or a bone cement (BMP-12, Wyeth Research). These “third party” carriers are problematic for several reasons. First, they require the surgeon to take additional steps to apply the carrier to the site of interest. Second, the carrier may be detrimental to healing. For example, recent studies have suggested that porcine small intestinal submucosa, a collagen scaffold, may be detrimental to rotator cuff healing. Third, the carrier must be “sandwiched” between the tendon and bone in order to exert maximal effects at the healing interface. However, the healing is in part related to the amount of physical contact area between the tendon and bone. Consequently, placing a carrier between these tissues may disrupt the body's natural biologic healing response. Finally, the agent must not be washed off of the carrier during arthroscopic surgery when the surgical site is distended with fluid.

There have been several attempts to modify the suture anchor with a feature that delivers a drug or growth factor. For example, U.S. Pat. No. 6,689,153 discloses a coated anchoring device and/or suture. However, there are a number of disadvantages with this type of design. For example, the agent diffuses in all directions and not necessarily towards the tendon-bone interface since the coating is over the sides and bottom tip of the suture. In addition, since the anchor is relatively small, and therefore limited in the amount of agent that can be applied to it, this can result in subtherapeutic doses of the agent at the tendon-bone interface.

U.S. Pat. No. 6,579,533 is directed to a bioasbsorbable drug delivery system; however, this type of device is limited to a specific synthetic bioabsorbable polymer that incorporates an antibiotic into its matrix. The patent mentions that the antibiotic polymer can be made into a suture anchor. As with the device of the '153 patent, this design suffers from the disadvantage that the antibiotic diffuses in all directions and not necessarily towards the tendon-bone interface.

U.S. patent application publication No. 2006/0178702 discloses an apparatus for attaching sutures. The apparatus can include a drug reservoir in the form of a blind hole in the side of the anchor. Once again, this design suffers from the disadvantage that the release location of the drug is not towards the bone/tendon interface where the drug or growth factor, etc. is most needed. Instead, the drug releases in side directions relative to the anchor. In addition, this side reservoir requires the anchor to have a specific design.

### SUMMARY

A surgical device for use in attaching a first member (e.g., soft-tissue, such as a tendon) to a second member (e.g., a bone) includes a suture anchor that has a body having a first end that includes a hollow anchor head; a pointed tip at an opposite second end; and a contoured outer surface. The anchor head has an opening to permit attachment of one or more sutures to the body. The suture anchor also includes a reservoir formed in the body that is open at the first end thereof and closed at an opposite second end. The reservoir is configured to store and release at least one of a drug, a therapeutic agent (such as an anesthetic), a growth factor, or a combination thereof from a top surface of the anchor body into a site (location) of an interface between the first and second members to promote healing or limit pain therebetween.

In another embodiment, a suture anchor for use in attaching a first member (e.g., tissue, such as a tendon) to a second member (e.g., bone) includes a body having a first end and an opposite pointed second end and a contoured outer surface and a reservoir formed in the body and being open at the first end of the body and closed at an opposite end that is closer to the second end. The reservoir is configured to store and release at least one of a drug, therapeutic agent, and a growth factor toward an interface between the first and second members. The suture anchor has a hollow head portion that extends from the first end of the body such that reservoir is accessible therethrough. The hollow head portion has an eyelet formed therein for receiving one or more sutures.

### BRIEF DESCRIPTION OF THE DRAWING FIGURES

The foregoing and other features of the present invention will be more readily apparent from the following detailed description and drawings figures of illustrative embodiments of the invention in which:

FIG. 1 is a top and side perspective view of anchor suture having a drug/growth factor delivery reservoir according to one exemplary embodiment of the present invention;

FIG. 2 is a side elevation view of the anchor suture of FIG. 1;

FIG. 3 is a top plan view of the anchor suture of FIG. 1;

FIG. 4 is a first cross-sectional view of the anchor suture of FIG. 1;

FIG. 5 is a second cross-sectional view of the anchor suture of FIG. 1;

FIG. 6 is a side elevation view of the anchor suture of FIG. 1 used in rotator cuff repair surgery; and

FIG. 7 is a close-up side elevation view of the anchor suture of FIG. 6.

### DETAILED DESCRIPTION OF PREFERRED EMBODIMENTS

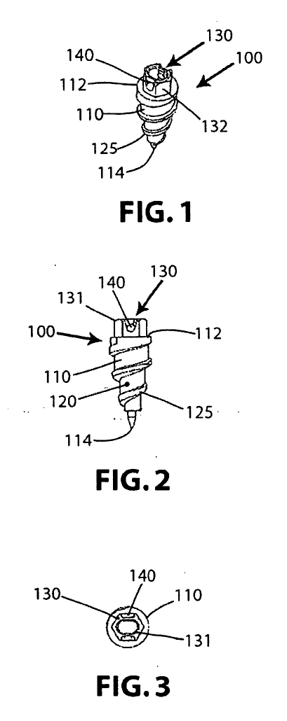

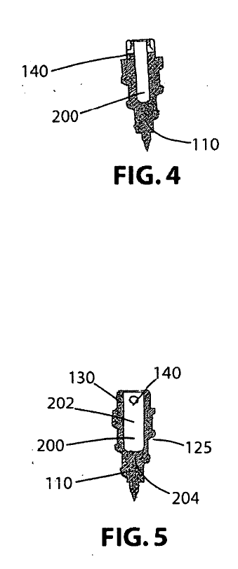

Referring to FIGS. 1-7, an anchoring device 100 according to one exemplary embodiment is illustrated and is particularly suited for tissue repair or to attach one object to another object (e.g., tissue to tissue, tissue to bone, and bone to bone). In addition, anchoring device 100 can be used to attach prosthetic or other materials foreign to the body to tissue and/or bone in the body.

The illustrated anchoring device 100 is in the form of a suture anchor that has an anchor body 110 that has a first end (proximal end) 112 and an opposing second end (distal end) 114. The anchor body 110 has an outer surface 120 that includes a securing means 125 that is configured to assist in anchoring the body 110 into an object, such as a bone. For example, in the illustrated embodiment, the securing means 125 is in the form of threads (e.g., spiral threads or screw edges) that serve to anchor the body 110 within bone as the anchor body 110 is implanted therein by using conventional tools, such as a driver. The distal end 114 is a sharp end (tip) for the initial insertion or further penetration into the tissue and/or bone.

The anchor body 110 can have a variable outer diameter in that the distal end 114 has an outer diameter that is less than the outer diameter of the proximal end 112. The anchor body 110 can have a tapered construction or it can have a stepped construction.

At or near the proximal end 112 of the anchor body 110, an anchor head 130 is formed and is configured to mate with the tool that is used to drive the anchor body 110 into the object to which it is implanted. The anchor head 130 is thus located above the proximal most thread 125. The anchor head 130 has an exterior surface 132 and it can be configured so that the tool (driver) engages the exterior surface 132 of the anchor head 130 in order to apply a driving force (torque) to the anchor 100 resulting in penetration and advancement of the anchor 100 into the object in which the anchor 100 is implanted. The illustrated anchor head 130 has a hexagonal shape and therefore, a complementary tool that is designed to engage hexagonal-shaped heads can be used to implant the anchor 100. It will be appreciated that other anchor head 130 designs can be used so long as they permit a tool to engage exterior surface 132 for driving and implanting the anchor 100.

In addition and in contrast to many existing suture anchor designs, the tool that is used to implant the anchor 100 into bone, etc. engages the exterior surface 132 of the anchor 100.

The anchor head 130 also has a means 140 for receiving and securing a suture (not shown). For example, the means 140 can be in the form of an eyelet 140 that secures the suture to just above the anchor body 110, not inside it. As shown in FIGS. 1-5, the eyelet 140 is formed through two opposing wall sections of the hollow anchor head 130. Thus, the portion of the suture that extends through the eyelet is positioned above the anchor body 110 since the eyelet 140 extends through the anchor head 130. It will be appreciated that the means 140 is not limited to being an eyelet; however, it can be another member. In other words, other methods and structures for attaching a suture to the anchor 100 can be equally used, including knotless designs.

In accordance with the present invention, the anchor 100 is formed to include a drug/growth factor reservoir 200 and more specifically, the anchor body 110 has a hollow construction. The reservoir 200 is thus formed by a bore that is formed through the anchor body 110 and is accessible through the hollow anchor head 130. The reservoir 200 is open at one end 202 that represents a top portion of the anchor body 110 where the anchor body 110 interfaces with the anchor head 130. The bore extends axially along the length of the anchor 100 and terminates in a closed end 204 that is spaced from the distal tip 114 of the anchor body 110. In other words, the reservoir 200 is encased by the anchor body 110 on its periphery and at the distal aspect of the anchor 100.

The hollow portion of the hollow head 130 is axially aligned with the opening to the reservoir 200 and therefore, the reservoir 200 is accessible through the hollow anchor head 130. A top surface 131 of the head 130 is open (perpendicular to the eyelet 140) to allow the reservoir 200 to be filled therethrough and conversely, this opening along the top surface 131 allows for transfer (diffusion) of the contents stored into the reservoir 200 to the location above the anchor 100 when it is implanted.

It will be appreciated that the shape of the reservoir 200 can be varied and in the illustrated embodiment, the reservoir 200 has a cylindrical shape. The size (volume) of the reservoir 200 can be varied depending upon the particular application and depending upon the dimensions of the anchor 100. The reservoir 200 is designed to apply sustained amounts of therapeutic agents to the healing soft-tissue-bone interface in order to improve healing, limit inflammation, or prevent pain. This results from the fact that the reservoir is open along the top of the anchor 100 as opposed to conventional anchors that incorporate a therapeutic agent along the sides and tip region of the anchor resulting in diffusion of the agent in all directions as opposed to supplying a concentrated amount of therapeutic agent to the location where it is most needed, namely, the soft tissue-bone interface above the anchor 100.

However, in one embodiment, the reservoir 200 is sized to receive about 50 microliter of volume inside the hollowed body of the anchor 100. The volume of the reservoir 200 is optimized (maximized) by several of the above-described design characteristics of the anchor 100 and in particular, the location of the eyelet 140 above the anchor body 110; the engagement of the driver to the outer surface of the anchor; and the anchor body has walls that are slightly thinner than conventional anchors.

It will be understood that any number of different types of substances can be disposed within the reservoir 200 depending upon the particular application that the anchor 100 is being used. In addition, the material characteristics of the substance can also vary in that the substance can be a liquid, a gel, a semi-solid, a slurry, etc.

Several drugs and growth factors can be used in the suture anchor 100 to promote healing, limit inflammation and control pain. These include, but are not limited to, antibiotics; local anesthetics, such as bupivicaine and lidocaine; anti-inflammatories; and growth factors, such as bone morphogenetic proteins (BMPs), platelet-derived growth factors (PDGF), basic fibroblast growth factor (bFGF), growth hormone (GH), insulin growth factor (IGF), transforming growth factor (TGF), and hepatocyte growth factor (HGF), etc. The reservoir 200 can be prefilled with a particular drug, growth factor, therapeutic agent (and labeled as such) or the reservoir 200 can be left empty and surgeons can place a particular substance into the reservoir. This permits the surgeon to tailor make the composition of the substance that is disposed within the reservoir 200. For example, based on a particular patient's needs, the surgeon can mix two or more compositions together and then add them to the reservoir 200.

In one embodiment, a sustained release drug delivery gel is stored in the reservoir 200. The gel can be formed of, but is not limited to, the following compositions: hydrogels, microspheres, lipospheres, collagen sponges, fibrin glue, bone cements, ceramics, or polymers, etc. The drug or growth factor can be embedded into any of these materials and then placed in the reservoir 200 of the anchor body 110. Alternatively, the drug or growth factor can placed in the reservoir 200 in a liquid form which is then allowed to diffuse through a permeable membrane (not shown) that covers the top of the reservoir 200.

In yet another embodiment, the anchor body 110 can contain multiple reservoirs 200 that each releases a different drug or therapeutic agent. For example, the multiple reservoirs 200 that open along a common plane (e.g., the interface between the head 130 and body 110) can be formed and each can be filled by access through the hollow anchor head 130.

It will be appreciated that for each drug or growth factor, the delivery system can be custom-made such that the release profile is clinically appropriate. For example, there is some evidence that PDGF applied at the time of surgery may not improve healing in a rabbit patellar tendon defect model. However, the delivery of PDGF on day seven (7) following surgery does improve healing in terms of histology and biomechanical testing. The delivery system, according to the present invention, used to carry PDGF must ensure that the drug is still being released on post-operative day 7. On the other hand, marcaine (a local anesthetic used for post-operative pain control) or an anti-inflammatory would need to be released immediately to achieve their therapeutic goals. The use of multiple drugs or growth factors into a single drug delivery carrier can, in some instances, dramatically improve soft tissue to bone healing. In addition, the temporal release of these factors can also influence healing. This can be addressed by incorporating growth factors into different microspheres with varying release profiles or creating multiple reservoirs 200 in the anchor body 110 that are covered with resorbable membranes of varying resorption profiles or simply with varying thicknesses to control the temporal elution of growth factors. This configuration would cause the release of one growth factor early in the healing process, and another later on. It will be understood that it is preferred and is possible with the present invention to adjust the composition of the drug delivery system to meet the specific needs of each drug or growth factor.

The anchor 100 is constructed to be compatible with a number of agents, and combinations thereof. The anchor body 110 can be formed of any biocompatible material, such as stainless steel, titanium, various polymers, collagen, allograft bone, or various other bioabsorbable materials, such as PLGA and PLLA. Each of these materials can also be either coated or embedded with the drug or growth factor.

The sutures can also be made of any biocompatible material, such as polymers, cellulose, protein-cellulose (silk), processed collagen (catgut), nylon, polypropylene, polyesters, Fiberware (Arthrex), Ethibond (Ethicon), etc. The anchor 100 can accommodate a single suture or multiple sutures. It will also be appreciated that the sutures can also be coated with the therapeutic agent.

As described above, the reservoir 200 in one embodiment has a volume of about 50 microliter for the following reasons. Theoretically, the growth factor or drug used with the present surgical device can be formulated at a concentration high enough to deliver an effective dose with the limited volume available. If this is not possible for some therapeutics, then multiple anchors 100 can be used, as is often the case clinically (e.g., double row suture technique (4 anchors), transosseous equivalent (2 anchors and 2 non-threaded pins)). Data on the ability of PDGF to improve periodontal bone healing in humans (available at www.biomimetrics.com) and a study that examined the ability of PDGF to improve soft tissue to bone healing in a rabbit knee medial collateral ligament repair model was extrapolated to show that a 50 microliter volume is a clinically useful and effective volume. BioMimetics, Inc. manufactures PDGF at a concentration of 0.3 mg/ml; however, formulations of 1 mg/ml have been used in clinical trials. The dose of PDGF that is FDA approved for periodontal bone defects is 150 micrograms. As a result when the more concentrated PDGF formulation of 1 mg/ml is used, three anchors 100 that each holds 50 microliters (containing 50 micrograms of PDGF) is needed to deliver the same dose of PDGF that is required to fill bone defects. However, other studies have shown that 20 micrograms are capable of improving soft tissue to bone healing. In applications where this is true, one anchor 100 can be used to sufficiently deliver a therapeutic dose.

It will therefore be understood that while, in one embodiment, the reservoir 200 has a 50 microliter volume, the reservoir 200 can be formed to have other volumes.

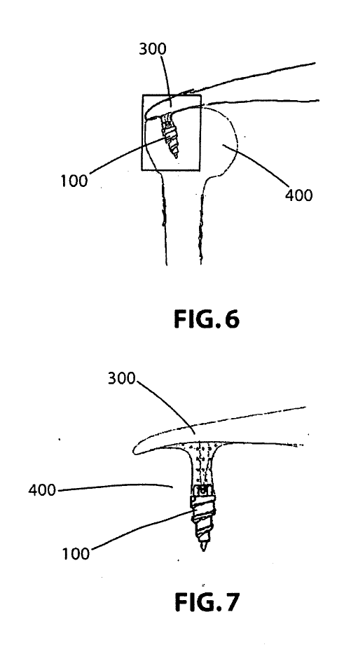

Referring to FIGS. 6-7 in which one application for the suture anchor 100 is shown. More specifically, the suture anchor 100 can be used in rotator cuff repair surgery. The rotator cuff, generally indicated at 300, is a group of four muscles that surround the humeral head 400 (ball of the shoulder joint). The muscles 300 are referred to as the “SITS” muscles: supraspinatus, infraspinatus, teres minor, and subscapularis. The muscles 300 function to provide rotation, elevate the arm, and give stability to the shoulder joint (glenohumeral joint).

During the surgical procedure, an arthroscope can be used and inserted near the shoulder joint through a small incision. The arthroscope is attached to a video monitor to allow the surgeon to see inside the shoulder joint. After properly prepping the area, the surgeon then inserts the suture anchors 100 into the bone 400 using a driver or the like as previously described. As shown, the suture anchor 100 is buried within the bone 400. The sutures are attached to the eyelet 140 of the suture anchor 100. The sutures can be woven through the torn tendon to assist in stabilizing and reattaching the torn tendon 300 to the bone 400.

As shown in FIG. 7, the suture anchor 100 is positioned in the bone 400 with the anchor head 130 facing the tendon and therefore, since the opening of the reservoir 200 faces the soft-tissue-bone interface, the released drug, growth factor or therapeutic agent is released at this important interface, thereby promoting improved healing at the interface.

The anchor 100 is configured to deliver a therapeutic drug or growth factor to the site of healing in contrast to conventional anchor/drug devices where the direction of drug released was not tailored. In other words, the anchor 100 has the mechanical features of conventional suture anchors; however, the anchor 100 incorporated a drug or growth factor delivery system into the body 110 of the anchor 100. This permits the surgeon to apply the agent without any additional steps to the procedure since it will be delivered when the suture anchor 100 is placed. The drug/growth factor is then released from the top of the anchor 100 into the site of the tendon-bone healing without having to place a “third party” carrier between these tissues. This will maximize the tendon-to-bone contact area while still delivering the agent to the area of healing. Moreover, the carrier will be shielded from most of the fluid in the joint since it will be covered on the periphery and distal surface by the body of the anchor. This should limit premature elution of the agent during arthroscopic-procedures.

It will also be understood that while FIGS. 6-7 illustrate the use of the anchor 100 in rotator cuff surgery to release growth factor(s) stored in the reservoir 200 to improve bone-tendon healing, the anchor 100 can be used in other application besides this one. For example, therapeutic agent can be an anti-inflammatory, such as an NSAID, a cytokine inhibitor (e.g., inflixamab, etanercept), or an anti-inflammatory cytokine (e.g., IL-10). Moreover, the therapeutic agent can also be an antibiotic to prevent or treat infection, or a local anesthetic to improve post-operative pain relief. In addition to rotator cuff surgery, the suture anchor 100 can be used for any procedure that calls for soft tissue to bone fixation. Such surgeries, include but are not limited to: medial collateral ligament repair, shoulder and hip labral repairs, biceps tenodesis, deltoid ligament repairs, triangular fibrocartilage complex repairs, lateral ulnar collateral ligament repair, etc. Also, as discussed above, the reservoir 200 can be left empty, thereby permitting the surgeon to select the precise contents thereof for any given application.

While exemplary drawings and specific embodiments of the present invention have been described and illustrated, it is to be understood that the scope of the present invention is not to be limited to the particular embodiments discussed. Thus, the embodiments shall be regarded as illustrative rather than restrictive, and it should be understood that variations may be made in those embodiments by workers skilled in the art without departing from the scope of the present invention as set forth in the claims that follow, and equivalents thereof. In addition, the features of the different claims set forth below may be combined in various ways in further accordance with the present invention.

What is claimed is:

1. A suture anchor for use in attaching a first member to a second member comprising:
a body having a first end and an opposite pointed second end and a contoured outer surface;
a reservoir formed in the body and being open at the first end of the body and closed at an opposite end that is closer to the second end, the reservoir being configured to store and release at least one of a drug, therapeutic agent, and a growth factor toward an interface between the first and second members; and
a hollow head portion that extends from the first end of the body such that an entrance to the reservoir is accessible therethrough, the hollow head portion having an eyelet formed therein for receiving one or more sutures.

2. The suture anchor of claim 1, wherein the first member comprises soft tissue and the second member comprises a bone.

3. The suture anchor of claim 2, wherein the first member comprises a tendon.

4. The suture anchor of claim 1, wherein the anchor body and anchor head are formed of a biocompatible material.

5. The suture anchor of claim 4, wherein the biocompatible material includes at least one of a stainless steel, a titanium, a polymer, a bone, or an absorbable.

6. The suture anchor of claim 5, wherein the polymer includes at least one of a polyester, a nylon, a poly lactic acid (PLA), a poly-L lactic acid (PLLA), or a poly glycolic acid (PGA).

7. The suture anchor of claim 1, wherein the contoured outer surface includes spiral threads.

8. The suture anchor of claim 1, wherein the anchor head is configured so that a tool can engage outer surfaces thereof to drive and implant the suture anchor into the second member.

9. The suture anchor of claim 8, wherein the anchor head has a hexagonal shape.

10. The suture anchor of claim 1, wherein the eyelet is formed above an interface between the anchor body and the anchor head.

11. The suture anchor of claim 1, wherein the reservoir comprises a bore formed in the anchor body along its length and from the first end thereof.

12. The suture anchor of claim 11, wherein the bore has a cylindrical shape.

13. The suture anchor of claim 1, wherein a width of the anchor head is less than a width of the first end of the anchor body.

14. The suture anchor of claim 1, wherein the reservoir has a volume of about 50 microliter.

15. The suture anchor of claim 1, wherein at least one of a drug, therapeutic agent, and a growth factor is in liquid form and the anchor further includes a permeable membrane that covers the open first end of the reservoir.

16. The suture anchor of claim 15, wherein the membrane is formed of a resorbable material.

17. The suture anchor of claim 1, wherein the at least one of a drug, therapeutic agent, and a growth factor comprises growth factors that are incorporated into different microspheres with varying release profiles.

18. The suture anchor of claim 1, further including at least one additional reservoir formed in the anchor body, each of the reservoirs being separate from one another.

19. The suture anchor of claim 18, wherein the reservoirs are covered with resorbable membranes of varying profiles.

20. The suture anchor of claim 18, wherein the reservoirs are covered with resorbable membranes of varying thicknesses.

21. The suture anchor of claim 1, wherein the at least one of a drug, therapeutic agent, and a growth factor improves healing, limit inflammation or prevent pain.

22. The suture anchor of claim 21, wherein the at least one of a drug, therapeutic agent, and a growth factor is selected from the group consisting of: antibiotics; local anesthetics, such as bupivicaine and lidocaine; anti-inflammatories; and growth factors, such as bone morphogenetic proteins (BMPs), platelet-derived growth factors (PDGF), basic fibroblast growth factor (bFGF), growth hormone (GH), insulin growth factor (IGF), transforming growth factor (TGF), and hepatocyte growth factor (HGF).

23. The suture anchor of claim 1, wherein a hollow interior section of the anchor head is accessible through an opening along a top surface of the anchor head and is axially aligned with the reservoir.

24. A surgical device for use in attaching a first member to a second member comprising:
a suture anchor including:
a body having a first end that includes a hollow anchor head; a pointed tip at an opposite second end; and a contoured outer surface, the anchor head having an opening to permit attachment of one or more sutures to the body; and
a reservoir formed in the body and being open at the first end thereof and closed at an opposite end, the reservoir being configured to store and release at least one of a drug, a therapeutic agent, and a growth factor from a top surface of the anchor body into a site of an interface between the first and second members to promote healing therebetween.

25. The surgical device of claim 24, wherein the first member comprises soft-tissue and the second member comprises a bone.

26. The surgical device of claim 24, wherein the top surface of the anchor body includes an opening that forms an entrance into the reservoir to permit the at least one of a drug, therapeutic agent, and a growth factor to migrate through the opening to the site.

27. The surgical device of claim 24, wherein the body and anchor head are an integral, unitary structure.

28. The surgical device of claim 24, wherein the reservoir stores a sustained release drug delivery gel, the gel being formed of a composition selected from the group consisting of: hydrogels, microspheres, lipospheres, collagen sponges, fibrin glue, bone cements, ceramics, and polymers; the drug, growth factor or therapeutic agent being embedded into the composition.

29. A method for delivering a drug, a growth factor or a therapeutic agent to a location where soft tissue contacts a bone comprising the steps of:
providing a suture anchor including a body having a pointed distal tip, a contoured outer surface, and an anchor head portion that includes an opening to permit attachment of one or more sutures to the body, the body having a reservoir formed therein and being open along a top surface of the anchor;
disposing a drug, a therapeutic agent, a growth factor, or a combination thereof in the reservoir; and
implanting the suture anchor in the bone such that the top surface faces the location where the soft tissue contacts the bone resulting in the drug, therapeutic agent, or growth factor being released from the top surface of the suture anchor and migrating in a direction toward an interface between the soft tissue and bone.
